(19) United States
(12) Patent Application Publication
(10) Pub. No.: US 2022/0396778 A1
(43) Pub. Date: Dec. 15, 2022

(54) Novel RNA Composition and Production Method for Use in iPS Cell Generation

(71) Applicants: Shi-Lung LIN, Arcadia, CA (US); Sam LIN, Arcadia, CA (US); Chun-Hung LIN, Taipei (TW)

(72) Inventors: Shi-Lung LIN, Arcadia, CA (US); Sam LIN, Arcadia, CA (US); Chun-Hung LIN, Taipei (TW)

(21) Appl. No.: 17/648,340

(22) Filed: Jan. 19, 2022

Related U.S. Application Data
(63) Continuation-in-part of application No. 17/489,357, filed on Sep. 29, 2021.
(60) Provisional application No. 63/209,969, filed on Jun. 12, 2021.
(60) Provisional application No. 63/210,988, filed on Jun. 15, 2021.
(60) Provisional application No. 63/212,657, filed on Jun. 19, 2021.
(60) Provisional application No. 63/222,398, filed on Jul. 15, 2021.
(60) Provisional application No. 63/270,034, filed on Oct. 20, 2021.
(60) Provisional application No. 63/280,226, filed on Nov. 17, 2021.

(51) Int. Cl. C12N 5/074; C12N 15/113; C12N 9/12; A61K 31/713; A61K 38/45

(52) U.S. Cl. CPC C12N 5/0696; C12N 15/113; C12N 9/127; C12Y 207/07048; A61K 31/713; A61K 38/45; C12N 2310/141; C12N 2320/31; C12N 2501/65; C12N 2506/09; C12N 2506/115

(57) ABSTRACT

This invention generally relates to a novel RNA composition and its production method useful for generating and expanding induced pluripotent stem cells (iPS cells; iPSC) as well as adult stem cells (ASC). The RNA composition so defined can be used for producing not only non-transgenic but also tumor-free iPS cells. The defined RNA composition contans at least two types of different RNA constructs; one is “miR-302 precursor RNA (pre-miR-302)” and the other is “RNA-dependent RNA polymerase (RdRp)” mRNA. Both of pre-miR-302 and RdRp mRNA contain highly structured RNA comformations, such as hairpin and stem-loop structures. To produce highly structured RNAs, a novel PCR-IVT methodology has been developed and used with a specially designed RNA polymerase-helicase mixture activity.

### CROSS-REFERENCE TO RELATED APPLICATIONS

The present invention claims priority to U.S. Provisional Patent Application No. 63/209,969 filed on Jun. 12, 2021, which is entitled “Novel mRNA Composition and Production for Use in Anti-Viral and Anti-Cancer Vaccines”. The present invention also claims priority to U.S. Provisional Patent Applications No. 63/210,988 filed on Jun. 15, 2021, Ser. No. 63/212,657 filed on Jun. 19, 2021, and No. 63/222,398 filed on Jul. 15, 2021, all of which are entitled “Novel mRNA Composition and Production Method for Use in Anti-Viral and Anti-Cancer Vaccines”. The present invention further claims priority to U.S. Provisional Patent Applications No. 63/270,034 filed on Oct. 20, 2021, and No. 63/280,226 filed on Nov. 17, 2021, both of which are entitled “Novel RNA Composition and Production Method for Use in iPS Cell Generation”. The present application is a continuation-in-part application of the U.S. patent application Ser. No. 17/489,357 filed on Sep. 29, 2021, which is entitled “Novel mRNA Composition and Production Method for Use in Anti-Viral and Anti-Cancer Vaccines”. The contents of each application are hereby incorporated by reference in their entirety.

### FIELD OF INVENTION

This invention generally relates to a novel RNA composition and its production method useful for generating and expanding induced pluripotent stem cells (iPS cells; iPSC) as well as adult stem cells (ASC). The RNA composition so defined can be used for producing not only non-transgenic but also tumor-free iPS cells. The defined RNA composition contans at least two types of different RNA constructs; one is “miR-302 precursor RNA (pre-miR-302)” and the other is “RNA-dependent RNA polymerase (RdRp)” mRNA. Both of pre-miR-302 and RdRp mRNA contain highly structured RNA comformations, such as hairpins and stem-loops. To produce highly structured RNAs, a novel PCR-IVT methodology has been developed and used with a specially designed RNA polymerase-helicase mixture activity.

### BACKGROUND

Induced pluripotent stem cells (iPS cells or iPSCs) were first reported by Yamanaka et al. in 2006, using three or four different protein transcription factors (U.S. Pat. Nos. 8,048,999 and 8,058,065 to Yamanaka et al.; Takahashi K and Yamanaka S, Cell 126:663-676, 2006). Alternatively, soon after that, Lin et al. first used microRNA (miRNA) miR-302 to reprogram human somatic cells into iPSCs in 2007 (European Patent No. EP2198025B1 and U.S. Pat. No. 9,567,591 to Lin et al.). Using miR-302, Lin et al. generated tumor-free iPSCs from not only normal tissue cells but also cancerous cells (Lin et al., RNA 14:2115-2124, 2008; Lin et al., Cancer Res. 70:9473-9482, 2010; Lin et al., Nucleic Acids Res. 39:1054-1065, 2011; Lin S L and Ying S Y, MicroRNA Protocols, 2nd Ed. Chapter 23, pp 295-324, Springer Publishers press, New York, 2012). Later in 2011, Lin et al. further revealed the mechanism underlying this miR-302-mediated somatic cell reprogramming event (Lin et al., Nucleic Acids Res. 39:1054-1065, 2011; Lin S L, Stem Cells 29:1645-1649, 2011). In addition to Lin's findings in 2011, two other independent research groups, Miyoshi et al. and Anokye-Danso et al., also simultaneously confirmed the essential role of miR-302 in iPSC generation (Miyoshi et al., Cell Stem Cell 8:633-638, 2011; Anokye-Danso et al., Cell Stem Cell 8:376-388, 2011). Eventually, all these major prior studies and inventions open up a whole new avenue for further research and development of many other following RNA-mediated iPSC-technologies.

As demonstrated in the cited prior studies. Lin et al. had identified that the reprogramming efficiency of iPSC generation is determined by the intracellular levels of miR-302 concentration (Lin et al., Nucleic Acids Res. 39:1054-1065, 2011). The miR-302 concentration level required for achieving complete iPSC formation is about ≥1.1˜1.3 folds of that level found in human embryonic stem cell (ESC) lines WA01-H1 or WA09-H9. However, the efficiency of current non-viral vector-based delivery of miR-302 or miR-302-mimic siRNA into somatic cells is hard to control and usually lower than the miR-302 concentration level required for achieving complete reprogramming. Hence, the addition of some other kinds of microRNAs like miR-369 or miR-367 may be needed for enhancing the reprogramming efficiency (Miyoshi et al., Cell Stem Cell 8:633-638, 2011; Anokye-Danso et al., Cell Stem Cell 8:376-388, 2011), because ma-367 and miR-369 can also silence some human tumor-suppressor genes, such as p57(kip2) and p63, the resulting iPSCs often show higher tumorigenecity than that of iPSCs reprogrammed by using only miR-302. As a result, a method of increasing the delivery and/or reprogramming efficiency or raising the intracellular miR-302 expression level is highly, desirable for overcoming such a tumorigenecity problem.

Not only that, another problem of miR-302-mediated iPSC generation is due to the highly structured conformations of miR-302 precursor RNA (pre-miR-302), which is very difficult to be produced by using conventional enzymatic or machinery synthesis methods. Prior mRNA/pre-miRNA production methods are mainly dependent on polymerase chain reaction and in vitro transcription (PCR-IVT) (U.S. Pat. Nos. 7,662,791, 8,080,652, 8,372,969, and 8,609,831 to Lin et al.); yet, these prior PCR-IVT methods are not designed to overcome the low production rate of highly structured RNAs. As depicted in FIG. 1, Lin's methods first use PCR and/or reverse transcription (RT) to incorporate a specific RNA promoter-primer into the resulting PCR products in order to generate promoter-driven DNA templates for IVT. Next, an IVT reaction is performed to produce and amplify desired RNA molecules from the DNA templates (Lin et al, Methods Mol Biol. 221:93-101, 2003). This PCR-IVT reaction can be reiterated multiple cycles for further amplifying the desired RNA molecules. After that, the desired RNAs can be transfected into targeted cells to generate piwi-interacting RNAs (piRNA) for silencing certain specific genes (U.S. Pat. Nos. 8,372,969 and 8,609,831 to Lin et al.). Nevertheless, although Lin's methods had been successfully used to produce mRNAs and piRNAs for eliciting at least a specific biological and/or gene silencing effect, these mRNAs and piRNAs so produced are not highly structured RNAs. Therefore, Lin's PCR-IVT methods did not disclose an effective way to overcome the low efficiency problem of highly structured RNA production in vitro.

To solve the low efficiency problem of highly structured RNA production, another kind of methods using plasmid-driven RNA expression in prokaryotes (i.e. bacteria) had been developed by Lin et al. (U.S. Pat. Nos. 9,637,747 and 9,783,811 to Lin et al.). These prokaryote-produced RNAs may contain one or multiple hairpin-like stem-loop structures, including pre-miR-302 cluster RNAs. In this approach, a chemical transcription inducer needs to be added into the medium of prokaryotic cell culture to overcome the problem of intrinsic transcription termination caused by hairpin RNAs (McDowell et al., Science 266:822-825, 1994), resulting in a marked increase of hairpin-like RNA production. Nevertheless, it is also noted that this plasmid-driven RNA expression methodology requires to use prokaryotic cells and the RNA production is limited in prokaryotes.

Since increasing the intracellular concentration of highly structured pre-miR-302 constructs for achieving high-throughput iPSC generation is one of key novelties of the present invention, it is herein required to have a methodology for not only overcoming the low efficiency problem of pre-miR-302 production but also raising the delivery/reprogramming efficiency of pre-miR-302 in targeted cells. Unfortunately, none of prior PCR-IVT and iPSC generation methods, not even in combination, can fulfill this goal. Therefore, a novel intracellular pre-miR-302 production method for inducing and enhancing iPSC generation is highly desired.

### SUMMARY OF THE INVENTION

The present invention is a method of inducing and enhancing induced pluripotent stem cell (iPSC) generation using a novel mechanism of intracellular miR-302 precursor RNA (pre-miR-302) generation stimulated by an artificially added RNA-dependent RNA polymerase (RdRp) amplification activity. The RdRp is preferably isolated and/or modified from RNA viruses, such as coronavirus and/or hepatitis C virus. Since using conventional transfection methods is difficult to deliver sufficient pre-miR-302 required for achieving complete reprogramming of somatic cells to form iPSCs, the present invention herein adopts a novel intracellular RdRp-mediated amplification mechanism to overcome this transfection/delivery problem. Due to this novel RdRp-mediated amplification mechanism, even only a small amount of delivered pre-miR-302 can be amplified at least forty to over thousand folds in the transfected cells, so as to provide sufficient intracellular pre-miR-302 concentrations for inducing and enhancing complete somatic cell reprogramming to form iPSCs. Conceivably, the same RdRp-mediated RNA amplification mechanism can be used to produce and amplify other various kinds of pre-miRNAs and mRNAs carrying at least an RdRp binding site, particularly viral, pathogenic antigen RNAs and/or known functional RNAs/mRNAs, which are useful for developing anti-viral and/or anti-disease/-cancer vaccines and medicines.

It is not reasonable for an ordinary skillful person in the art to anticipate the amplification of pre-miR-302 in somatic cells because somatic cells contain neither pre-miR-302 nor RdRp. miR-302 is one of major standard embryonic stem cell (ESC) markers exclusively expressed in pluripotent stem cells, such as human ESCs and iPSCs. On the other hand, RdRp mainly exists in RNA viruses and is required for RNA viral multiplication. Hence, under a natural condition, reprogramming can not occur in somatic cells even after infected by RNA viruses. Yet, the present invention first discloses that somatic cell reprogramming can easily occur when the somatic cells are pre-transfected with a specially designed pre-miR-302 construct before RNA viral infection. During RNA viral infection, viral RdRp does not randomly amplify all intracellular RNAs at the same rate, but more preferably amplifies those RNAs carrying at least an RdRp-binding site, such as viral RNAs. This amplification rate difference can be over several hundred to thousand folds. Using the same mechanism, our designed pre-miR-302 constructs herein carry at least an isolated RdRp-binding site. In this way, RdRp amplifies the designed pre-miR-302 constructs over forty to thousand folds in the transfected cells, resulting in a marked increase of intracellular miR-302 concentration, of which the level is sufficient for inducing and enhancing complete iPSC generation.

The general structure of our specially designed pre-miR-302 constructs contain at least a hairpin-like miR-302 precursor (pre-miR-302) sequence flanked with at least an RdRp binding site (equivalent to promoter and/or enhancer) in its either 5′-end or 3′-end region, or both. As shown in FIG. 2, in order to efficiently initiate the desired RdRp activity, the designed pre-miR-302 construct must contain at least a 5′-end forward RdRp initiation/binding site or at least a 3′-end reverse RdRp initiation/binding site, or both. Before and after the initiation/binding sites, there may further contain a short about 1- to 55-nucleotide sequence of adenosine/uridine-rich (A/U-rich) motif, which is resulted from the desired RdRp extension activity. The 5′-end RdRp initiation/binding site contains at least a sequence of either 5′-AU(G/C)(U/-)G(A/U)-3′ (i.e. 5′-AUSUGW-3′; SEQ.ID.NO.1) or 5′-U(C/-)(U/A)C(U/C)(U/A)A-3′ (i.e. 5′-UCWCYWA-3′; SEQ.ID.NO.2), or both. For example, the 5′-end RdRp initiation/binding site is preferably selected from at least a sequence containing 5′-AUCUGU-3′ (SEQ.ID.NO.3), 5′-UCUCUAA-3′ (SEQ.ID.NO.4), 5′-UCUCCUA-3′ (SEQ.ID.NO.5), and/or 5′-UUCAA-3′ (SEQ.ID.NO.6), or a combination thereof. On the other hand, the 3′-end RdRp initiation/binding site contains at least a sequence of either 5′-(U/A)C(A/-)(C/G)AU-3′ (i.e. 5′-WCASAU-3′; SEQ.ID.NO.7) or 5′-U(A/U)(A/G)G(A/U)(G/-)A-3′ (i.e. 5′-UWRGWR-3′; SEQ.ID.NO.8), or both. For example, the 3′-end RdRp initiation/binding site is preferably selected from at least a sequence containing 5′-ACAGAU-3′ (SEQ.ID.NO.9), 5′-UUAGAGA-3′ (SEQ.ID.NO.10), 5′-UAGGAGA-3′ (SEQ.ID.NO.11), and/or 5′-UUGAA-3′ (SEQ.ID.NO.12), or a combination thereof. Notably, these defined RdRp initiation/binding sites were exclusively identified by the inventors of the present invention. Due to this special novel design, the desired RdRp activity can efficiently transcribe and amplify either the sense or antisense strand, or both, of the designed pre-miR-302 constructs in cells in vitro, ex vivo as well as in vivo.

In one preferred embodiment, the specially designed RNA (i.e, pre-miR-302 and/or RdRp mRNA, or other RNAs/mRNAs) construct contains at least an RdRp-binding site in both of its 5′- and 3′-end regions. Since both ends of the designed RNA construct carry at least an RdRp-binding site for RNA amplification with RdRp, the sense strand RNA can be used to amplify its complementary antisense RNAs (cRNA or aRNA), while the antisense strand RNA can be used to amplify the sense RNAs as well, so as to form an amplification cycle of both of the sense and antisense strand RNAs and thus resulting in a maximal amplification rate of the designed RNA construct. Also, the resulting sense and antisense strands of amplified RNAs may further form double-stranded RNAs in cells after transfection, facilitating the formation of siRNAs, shRNAs, miRNAs, and/or piRNAs of the designed RNA construct.

Alternatively, in another preferred embodiment, the specially designed RNA construct contains at least an RdRp-binding site in its either 5′-end or 3′-end region. In this way, we can selectively amplify either the sense or antisense strand of the designed RNA construct, leading to more specific amplification of the desired strand of the designed RNA construct. Particularly, this approach is useful for generating and amplifying either the mRNA or the antisense RNA (aRNA) of a specific functional protein in cells after transfection, so as to either produce/increase or inhibit/decrease the generation of the functional protein in the transfected cells.

Furthermore, the designed pre-miR-302 constructs may contain one or more hairpin-like miR-302 precursor sequences preferably selected from 5′-CCUUUGCUUU AACAUGGGGG UACCUGCUGU GUGAAACAAA AGUAAGUGCU UCCAUGUUUC AGUGGAGG-3′ (68-nt; SEQ.ID.NO.13), 5′-GCUCCCUUCA ACUUUAACAU GGAAGUGCUU UCUGUGACUU UAAAAGUAAG UGCUUCCAUG UUUUAGUAGG AGU-3′ (73-nt; SEQ.ID.NO.14), 5′-CCACCACUUA AACGUGGAUG UACUUGCUUU GAAACUAAAG AAGUAAGUGC UUCCAUGUUU UGGUGAUGG-3′ (69-nt; SEQ.ID.NO.15), and/or 5′-CCUCUACUUU AACAUGGAGG CACUUGCUGU GACAUGACAA AAAUAAGUGC UUCCAUGUUU GAGUGUGG-3′ (68-nt; SEQ.ID.NO.16), or a combination thereof. Between two individual miR-302 precursor sequences, there may further contain a 50˜500-nucleotide spacer sequence for preventing hairpin tangle formation. Conceivably, these miR-302 precursor sequences can be replaced by miR-302-mimic short-interfering RNA (siRNA) and/or small hairpin RNA (shRNA) sequences for eliciting the same functional purpose. Also, the uridine (U) content of these sequences can be replaced by pseudouridine and/or other modified nucleotides to increase the stability of the designed pre-miR-302 constructs as well as RdRp mRNA.

To efficiently produce these highly structured pre-miR-302 constructs and RdRp mRNA, we had developed a novel PCR-IVT methodology for overcoming the low efficiency problem of highly structured RNA generation (shown in U.S. patent application Ser. No. 17/489,357 to Lin et al.). Traditionally, it is not reasonable for an ordinary skillful person in the art to anticipate the efficient generation of highly structured RNAs in vitro because it is known that the presence of hairpin-like RNA structures greatly hinders RNA transcription. In fact, hairpin-like stem-loop structures are signals of intrinsic transcription termination for prokaryotic RNA polymerases (McDowell et al, Science 266:822-825, 1994). To solve this problem, the present invention adopts a novel IVT system with a mixture of RNA polymerase and helicase activities. The addition of helicase activity in IVT markedly reduces the secondary structures of both DNA templates and the resulting RNA products for far more efficiently producing highly structured RNAs. Accordingly, an improved buffer system is also required to maintain and enhance the efficiency of mixed RNA polymerase and helicase activities in IVT. Interestingly, although several prior studies had reported that helicase may be involved in prokaryotic transcription termination, the present invention however demonstrates a totally different functionality of helicase in RNA production during IVT.

For facilitating intracellular delivery/transfection in vitro, ex vivo or in vivo, the specially designed pre-miR-302 construct and RdRp mRNA can be mixed, conjugated, encapsulated and/or formulated with at least a delivery/transfection agent selected from, but not limited to, glycylglycerin-derived chemicals, liposomes, nanoparticles, liposomal nanoparticles (LNP), conjugating molecules, infusion/transfusion chemicals, gene gun materials, electroporation agents, transposons/retrotransposons, and a combination thereof.

After co-transfection of the designed pre-miR-302 construct and RdRp mRNA into desired target cells, iPSCs will quickly form within one to two weeks, depending on the reprogrammed cell types, via an induced biological process called somatic cell reprogramming. Lin's prior review article had already disclosed the underlying mechanism of this reprogramming event (Lin S L, Stem Cells 29:1645-1649, 2011). In experiments, the inventors have already tested this novel reprogramming methodology in both human normal and cancerous somatic cells, including isolated normal fibroblasts, isolated normal skin cells (in vitro as well as ex vivo), isolated human leukemia cells, human lung epithelial cell line BEAS-2B as well as cancerous lung NSCLC A549, breast adenocarcinoma MCF7, prostate carcinoma PC3, liver hepatocarcinoma HepG2, and skin melanoma Colo-829 cell lines. Hence, in view of high genomic similarity of these various human cells, the cell types capable of being reprogrammed into iPSCs include but not limited to cell substrates containing normal human tissue cells, somatic cells, diseased cells, tumor/cancer cells, skin-content-associated cells, and/or blood-content-associated cells, and a combination thereof.

Using the present invention, the inventors had gathered evidence for the success of iPSC generation in six areas: (1) markedly increased miR-302 expression in reprogrammed cells after co-transfection of pre-miR-302 and RdRp mRNA (FIG. 3), (2) elevated expression of standard ESC markers such as Oct3/4, Sox2 and Nanog found in reprogrammed cells 2-3 days after co-transfection of pre-miR-302 and RdRp mRNA (FIG. 4), (3) iPSC-derived embryoid body formation (FIG. 5), (4) global genomic DNA demethylation observed, similar to the status of a zygotic genome (FIG. 6), (5) over 92% high similarity of genome-wide gene expression patterns compared to those of human ESC WA01 (H1) and WA09 (H9) cell lines (FIG. 7), and (6) iPSC-derived teratoma formation containing tissues derived from all three embryonic germ layers (ectoderm, mesoderm and definitive endoderm) (FIG. 8). Furthermore, the inventors had also successfully used electroporation-based delivery methods to achieve similar iPSC generation results. Given that the function of miR-302 is known to reprogram both normal and cancerous tissue cells to tumor-free ESC-like iPSCs with a high >70% success rate, the findings of this novel invention may provide great benefits for designing and developing advanced applications in both stem cell and anti-cancer therapies.

The advantages of using the designed pre-miR-302 and RdRp mRNA mixture composition to induce iPSC generation include (1) no transgenic concerns, (2) no ethical concerns, (3) tumor-free grade high safety, (4) easy handling and preparation, (5) >70% iPSC formation efficiency, (7) stable and fast reprogramming efficiency within 1-2 weeks, and (8) multiple applications for use in in-vitro, ex-vivo, and/or in-vivo cell reprogramming. Further in view of our prior U.S. patent application Ser. No. 15/661,346 and Ser. No. 16/135,723 to Lin et al., the present invention may also be used to generate and expand adult stem cell (ASC) populations as well. Conceivably, the present invention is useful for designing and developing new pharmaceutical and therapeutic applications or devices, such as cell-based and/or reprogramming-associated therapies as well as medicines.

### A. Definitions

To facilitate understanding of the invention, a number of terms are defined below:

Nucleic Acid: a polymer of deoxyribonucleic acid (DNA) or ribonucleic acid (RNA), either single or double stranded.

Nucleotide: a monomeric unit of DNA or RNA consisting of a sugar moiety (pentose), a phosphate, and a nitrogenous heterocyclic base. The base is linked to the sugar moiety via the glycosidic carbon (1′ carbon of the pentose) and that combination of base and sugar is a nucleoside. A nucleoside containing at least one phosphate group bonded to the 3′ or 5′ position of the pentose is a nucleotide. DNA and RNA are consisted of different types of nucleotide units called deoxyribonucleotide and ribonucleotide, respectively.

Oligonucleotide: a molecule comprised of two or more monomeric units of DNA and/or RNA, preferably more than three, and usually more than ten. An oligonucleotide longer than 13 nucleotide monomers is also called polynucleotide. The exact size will depend on many factors, which in turn depends on the ultimate function or use of the oligonucleotide. The oligonucleotide may be generated in any manner, including chemical synthesis, DNA replication, RNA transcription, reverse transcription, or a combination thereof.

Nucleotide Analog: a purine or pyrimidine nucleotide that differs structurally from adenine (A), thymine (T), guanine (G), cytosine (C), or uracil (U), but is sufficiently similar to substitute for the normal nucleotide in a nucleic acid molecule.

Nucleic Acid Composition: a nucleic acid composition refers to an oligonucleotide or polynucleotide such as a DNA or RNA sequence, or a mixed DNA/RNA sequence, in either a single-stranded or a double-stranded molecular structure.

Gene: a nucleic acid composition whose oligonucleotide or polynucleotide sequence codes for an RNA and/or a polypeptide (protein). A gene can be either RNA or DNA. A gene may encode a non-coding RNA, such as small hairpin RNA (shRNA), microRNA (miRNA), rRNA, tRNA, snoRNA, snRNA, and their RNA precursors as well as derivatives. Alternatively, a gene may encode a protein-coding RNA essential for protein/peptide synthesis, such as messenger RNA (mRNA) and its RNA precursors as well as derivatives. In some cases, a gene may encode a protein-coding RNA that also contains at least a microRNA or shRNA sequence.

Primary RNA Transcript: an RNA sequence that is directly transcribed from a gene without any RNA processing or modification.

Precursor messenger RNA (pre-mRNA): primary RNA transcripts of a protein-coding gene, which are produced by eukaryotic type-II RNA polymerase (Pol-II) machineries in eukaryotes through an intracellular mechanism termed transcription. A pre-mRNA sequence contains a 5′-untranslated region (UTR), a 3′-UTR, exons and introns.

Intron: a part or parts of a gene transcript sequence encoding non-protein-reading frames, such as in-frame intron, 5′-UTR and 3′-UTR.

Exon: a part or parts of a gene transcript sequence encoding protein-reading frames (cDNA), such as cDNA for cellular genes, growth factors, insulin, antibodies and their analogs/homologs as well as derivatives.

Messenger RNA (mRNA): assembly of pre-mRNA exons, which is formed after intron removal by intracellular RNA splicing machineries (e.g. spliceosomes) and served as a protein-coding RNA for peptide/protein synthesis. The peptides/proteins encoded by mRNAs include, but not limited, enzymes, growth factors, insulin, antibodies and their analogs/homologs as well as derivatives.

Complementary DNA (cDNA): a single-stranded or double-stranded DNA that contains a sequence complementary to an mRNA sequence and does not contain any intronic sequence.

Sense: a nucleic acid molecule in the same sequence order and composition as the homologous mRNA. The sense conformation is indicated with a “+”, “s” or “sense” symbol.

Antisense: a nucleic acid molecule complementary to the respective mRNA molecule. The antisense conformation is indicated as a “—” symbol or with an “a” or “antisense” in front of the DNA or RNA, e.g., “aDNA” or “aRNA”.

Base Pair (bp): a partnership of adenine (A) with thymine (T), or of cytosine (C) with guanine (G) in a double stranded DNA molecule. In RNA, uracil (U) is substituted for thymine. Generally the partnership is achieved through hydrogen bonding. For example, a sense nucleotide sequence “5′-A-T-C-G-U-3” can form complete base pairing with its antisense sequence “5′-A-C-G-A-T-3”.

5′-end: a terminus lacking a nucleotide at the 5′ position of successive nucleotides in which the 5′-hydroxyl group of one nucleotide is joined to the 3′-hydroyl group of the next nucleotide by a phosphodiester linkage. Other groups, such as one or more phosphates, may be present on the terminus.

3′-end: a terminus lacking a nucleotide at the 3′ position of successive nucleotides in which the 5′-hydroxyl group of one nucleotide is joined to the 3′-hydroyl group of the next nucleotide by a phosphodiester linkage. Other groups, most often a hydroxyl group, may be present on the terminus.

Template: a nucleic acid molecule being copied by a nucleic acid polymerase. A template can be single-stranded, double-stranded or partially double-stranded, RNA or DNA, depending on the polymerase. The synthesized copy is complementary to the template, or to at least one strand of a double-stranded or partially double-stranded template. Both RNA and DNA are synthesized in the 5′ to 3′ direction. The two strands of a nucleic acid duplex are always aligned so that the 5′ ends of the two strands are at opposite ends of the duplex (and, by necessity, so then are the 3′ ends).

Nucleic Acid Template: a double-stranded DNA molecule, double-stranded RNA molecule, hybrid molecules such as DNA-RNA or RNA-DNA hybrid, or single-stranded DNA or RNA molecule.

Conserved: a nucleotide sequence is conserved with respect to a pre-selected (referenced) sequence if it non-randomly hybridizes to an exact complement of the pre-selected sequence.

Homologous or Homology: a term indicating the similarity between a polynucleotide and a gene or mRNA sequence. A nucleic acid sequence may be partially or completely homologous to a particular gene or mRNA sequence, for example. Homology may be expressed as a percentage determined by the number of similar nucleotides over the total number of nucleotides.

Complementary or Complementarity or Complementation: a term used in reference to matched base pairing between two polynucleotides (i.e. sequences of an mRNA and a cDNA) related by the aforementioned “base pair (bp)” rules. For example, the sequence “5′-A-G-T-3” is complementary to not only the sequence “5′-A-C-T-3” but also to “5′-A-C-U-3”. Complementation can be between two DNA strands, a DNA and an RNA strand, or between two RNA strands. Complementarity may be “partial” or “complete” or “total”. Partial complementarity or complementation occurs when only some of the nucleic acid bases are matched according to the base pairing rules. Complete or total complementarity or complementation occurs when the bases are completely or perfectly matched between the nucleic acid strands. The degree of complementarity between nucleic acid strands has significant effects on the efficiency and strength of hybridization between nucleic acid strands. This is of particular importance in amplification reactions, as well as in detection methods that depend on binding between nucleic acids. Percent complementarity or complementation refers to the number of mismatch bases over the total bases in one strand of the nucleic acid. Thus, a 50% complementation means that half of the bases were mismatched and half were matched. Two strands of nucleic acid can be complementary even though the two strands differ in the number of bases. In this situation, the complementation occurs between the portion of the longer strand corresponding to the bases on that strand that pair with the bases on the shorter strand.

Complementary Bases: nucleotides that normally pair up when DNA or RNA adopts a double stranded configuration.

Complementary Nucleotide Sequence: a sequence of nucleotides in a single-stranded molecule of DNA or RNA that is sufficiently complementary to that on another single strand to specifically hybridize between the two strands with consequent hydrogen bonding.

Hybridize and Hybridization: the formation of duplexes between nucleotide sequences which are sufficiently complementary to form complexes via base pairing. Where a primer (or splice template) “hybridizes” with target (template), such complexes (or hybrids) are sufficiently stable to serve the priming function required by a DNA polymerase to initiate DNA synthesis. There is a specific, i.e. non-random, interaction between two complementary polynucleotides that can be competitively inhibited.

Posttranscriptional Gene Silencing: a targeted gene knockout or knockdown effect at the level of mRNA degradation or translational suppression, which is usually triggered by either foreign/viral DNA or RNA transgenes or small inhibitory RNAs.

RNA Interference (RNAi): a posttranscriptional gene silencing mechanism in eukaryotes, which can be triggered by small inhibitory RNA molecules such as microRNA (miRNA), small hairpin RNA (shRNA) and small interfering RNA (siRNA). These small RNA molecules usually function as gene silencers, interfering with expression of intracellular genes containing either completely or partially complementarity to the small RNAs.

Gene Silencing Effect: a cell response after a gene function is suppressed, consisting but not limited of cell cycle attenuation, G0/G1-checkpoint arrest, tumor suppression, anti-tumorigenecity, cancer cell apoptosis, and a combination thereof.

Non-coding RNA: an RNA transcript that cannot be used to synthesize peptides or proteins through intracellular translation machineries. Non-coding RNA includes long and short regulatory RNA molecules such as microRNA (miRNA), small hairpin RNA (shRNA), small interfering RNA (siRNA) and double strand RNA (dsRNA). These regulatory RNA molecules usually function as gene silencers, interfering with expression of intracellular genes containing either completely or partially complementarity to the non-coding RNAs.

MicroRNA (miRNA): single-stranded RNAs capable of binding to targeted gene transcripts that have partial complementarity to the miRNA. MiRNA is usually about 17-27 oligonucleotides in length and is able to either directly degrade its intracellular mRNA target(s) or suppress the protein translation of its targeted mRNA, depending on the complementarity between the miRNA and its target mRNA. Natural miRNAs are found in almost all eukaryotes, functioning as a defense against viral infections and allowing regulation of gene expression during development of plants and animals.

Precursor MicroRNA (Pre-miRNA): hairpin-like single-stranded RNAs containing stem-arm and stem-loop regions for interacting with intracellular RNaseIII endoribonucleases to produce one or multiple microRNAs (miRNAs) capable of silencing a targeted gene or genes complementary to the microRNA sequence(s). The stem-arm of a pre-miRNA can form either a perfectly (100%) or a partially (mis-matched) hybrid duplexes, while the stem-loop connects one end of the stem-arm duplex to form a circle or hairpin-loop conformation. In the present invention, however, precursor of microRNA may also includes pri-miRNA.

Small interfering RNA (siRNA): short double-stranded RNAs sized about 18-27 perfectly base-paired ribonucleotide duplexes and capable of degrading target gene transcripts with almost perfect complementarity.

Small or short hairpin RNA (shRNA): single-stranded RNAs that contain a pair of partially or completely matched stem-arm nucleotide sequences divided by an unmatched loop or bubble oligonucleotide to form a hairpin-like structure. Many natural miRNAs are derived from small hairpin-like RNA precursors, namely precursor microRNA (pre-miRNA).

Vector: a recombinant nucleic acid composition such as recombinant DNA (rDNA) capable of movement and residence in different genetic environments. Generally, another nucleic acid is operatively linked therein. The vector can be capable of autonomous replication in a cell in which case the vector and the attached segment is replicated. One type of preferred vector is an episome, i.e., a nucleic acid molecule capable of extrachromosomal replication. Preferred vectors are those capable of autonomous replication and expression of nucleic acids. Vectors capable of directing the expression of genes encoding for one or more polypeptides and/or non-coding RNAs are referred to herein as “expression vectors” or “expression-competent vectors”. Particularly important vectors allow cloning of cDNA from mRNAs produced using a reverse transcriptase. A vector may contain components consisting of a viral or a type-II RNA polymerase (Pol-II or pol-2) promoter, or both, a Kozak consensus translation initiation site, polyadenylation signals, a plurality of restriction/cloning sites, a pUC origin of replication, a SV40 early promoter for expressing at least an antibiotic resistance gene in replication-competent prokaryotic cells, an optional SV40 origin for replication in mammalian cells, and/or a tetracycline responsive element. The structure of a vector can be a linear or circular form of single- or double-stranded DNA selected form the group consisting of plasmid, viral vector, transposon, retrotransposon, DNA transgene, jumping gene, and a combination thereof.

Promoter: a nucleic acid to which a polymerase molecule recognizes, perhaps binds to, and initiates RNA transcription. For the purposes of the instant invention, a promoter can be a known polymerase binding site, an enhancer and the like, any sequence that can initiate synthesis of RNA transcripts by a desired polymerase.

Restriction Site: a DNA motif for restriction enzyme cleavage including but not limited AatII, AccI, AfIII/III, AgeI, ApaI/LI, AseI, Asp718I, BamHI, BbeI, BclI/II, BgIII, BsmI, Bsp120I, BspHI/LU11I/120I, BsrI/BI/GI, BssHII/SI, BstBEUI/XI, ClaI, Csp6I, DpnI, DraI/II, EagI, Ecl136II, EcoRI/RII/47III/RV, EheI, FspI, HaeIII, HhaI, HinPI, HindIII, HinfI, HpaI/II, KasI, KpnI, MaeII/III, MfeI, MluI, MscI, MseI, NaeI, NarI, NcoI, NdeI, NgoMI, NotI, NruI, NsiI, Ppu10I, PstI, Pvul/II, RsaI, SacI/II, SaII, Sau3AI, SmaI, SnaBI, SphI, SspI, StuI, TaiI, TaqI, XbaI, XhoI, XmaI cleavage site.

Cistron: a sequence of nucleotides in a DNA molecule coding for an amino acid residue sequence and including upstream and downstream DNA expression control elements.

RNA Processing: a cellular mechanism responsible for RNA maturation, modification and degradation, including RNA splicing, intron excision, exosome digestion, nonsense-mediated decay (NMD), RNA editing, RNA processing, 5′-capping, 3′-poly(A) tailing, and a combination thereof.

Gene Delivery: a genetic engineering method selected from the group consisting of polysomal transfection, liposomal transfection, chemical (nanoparticle) transfection, electroporation, viral infection, DNA recombination, transposon insertion, jumping gene insertion, microinjection, gene-gun penetration, and a combination thereof.

Genetic Engineering: a DNA recombination method selected from the group consisting of DNA restriction and ligation, homologous recombination, transgene incorporation, transposon insertion, jumping gene integration, retroviral infection, and a combination thereof.

Tumor Suppression: a cellular anti-tumor and anti-cancer mechanism consisting but not limited of cell cycle attenuation, G0/G1-checkpoint arrest, tumor suppression, anti-tumorigenecity, cancer cell apoptosis, and a combination thereof.

Targeted Cell: a single or a plurality of human cells selected from the group consisting of a somatic cell, a tissue cell, a stem cell, a germ-line cell, a tumor cell, a cancer cell, a virus-infected cell, and a combination thereof.

Cancerous Tissue: a neoplastic tissue derived from the group consisting of skin cancer, prostate cancer, breast cancer, liver cancer, lung cancer, brain tumor/cancer, lymphoma, leukemia and a combination thereof.

Antibody: a peptide or protein molecule having a pre-selected conserved domain structure coding for a receptor capable of binding a pre-selected ligand.

Pharmaceutical and/or therapeutic Application: a biomedical utilization and/or apparatus useful for stem cell generation, stem cell research and/or therapy development, cancer therapy, disease treatment, wound healing and tissue regeneration treatment, high-yield production of drug and/or food supplies, and a combination thereof.

Prokaryote or Prokaryotic Cell: an one-cell organism that lacks a distinct membrane-bound nucleus and has its genetic materials in the form of a continuous strand of DNA, such as bacteria.

Eukaryote or Eukaryotic Cell: an one-cell or multiple-cell organism whose cells contain a nucleus and other structures (organelles) enclosed within membranes, such as yeast, plant and animal cells.

Transcription Inducer: a chemical agent that can induce and/or enhance eukaryotic RNA and/or gene transcription from a eukaryotic pol-2 or pol-2 equivalent promoter in prokaryotic cells. For example, a transcription inducer contains, but not limited, a chemical structure similar to 3-morpholinopropane-1-sulfonic acid (MOPS), ethanol and/or glycerin, as well as their functional analogs, such as mannitol, 2-(N-morpholino)ethanesulfonic acid (MES) and 4-(2-hydroxyethyl)-1-piperazineethanesulfonic acid (HEPES), or a mixture thereof.

### B. Compositions and Applications

A novel RNA composition and its production method for use in induced pluripotent stem cell (iPSC) generation, comprising:

at least a miR-302 precursor RNA (pre-miR-302) construct and at least an RNA-dependent RNA polymerase (RdRp) mRNA, wherein the pre-miR-302 construct contains at least a 5′-end RdRp binding site or at least a 3′-end RdRp binding site, or both, and wherein the RdRp mRNA is isolated and/or modified from RNA virus, preferably from coronavirus or hepatitis C virus. Structurally, the 5′-end RdRp binding site contains at least a sequence of either 5′-AU(G/C)(U/-)G(A/U)-3′ (5′-AUSUGW-3′; SEQ.ID.NO.1) or 5′-U(C/-)(U/A)C(U/C)(U/A)A-3′ (5′-UCWCYWA-3′; SEQ.ID.NO.2), or both. For instance, the 5′-end RdRp binding site is preferably selected from at least an RNA sequence containing 5′-AUCUGU-3′ (SEQ.ID.NO.3), 5′-UCUCUAA-3′ (SEQ.ID.NO.4), 5′-UCUCCUA-3′ (SEQ.ID.NO.5), and/or 5′-UUCAA-3′ (SEQ.ID.NO.6), or a combination thereof. On the other hand, the 3′-end RdRp binding site contains at least a sequence of either 5′-(U/A)C(A/-)(C/G)AU-3′ (5′-WCASAU-3′; SEQ.ID.NO.7) or 5′-U(A/U)(A/G)G(A/U)(G/-)A-3′ (5′-UWRGWR-3′; SEQ.ID.NO.8), or both. For example, the 3′-end RdRp binding site is preferably selected from at least an RNA sequence containing 5′-ACAGAU-3′ (SEQ.ID.NO.9), 5′-UUAGAGA-3′ (SEQ.ID.NO.10), 5′-UAGGAGA-3′ (SEQ.ID.NO.11), and/or 5′-UUGAA-3′ (SEQ.ID.NO.12), or a combination thereof.

In addition, the pre-miR-302 construct contains at least a hairpin-like miR-302 precursor sequence preferably selected from 5′-CCUUUGCUUU AACAUGGGGG UACCUGCUGU GUGAAACAAA AGUAAGUGCU UCCAUGUUUC AGUGGAGG-3′ (SEQ.ID.NO.13), 5′-GCUCCCUUCA ACUUUAACAU GGAAGUGCUU UCUGUGACUU UAAAAGUAAG UGCUUCCAUG UUUUAGUAGG AGU-3′ (SEQ.ID.NO.14), 5′-CCACCACUUA AACGUGGAUG UACUUGCUUU GAAACUAAAG AAGUAAGUGC UUCCAUGUUU UGGUGAUGG-3′ (SEQ. ID.NO. 15), and/or 5′-CCUCUACUUU AACAUGGAGG CACUUGCUGU GACAUGACAA AAAUAAGUGC UUCCAUGUUU GAGUGUGG-3′ (SEQ.ID.NO.16), or a combination thereof. Between two individual miR-302 precursor sequences, there may further contain a 50˜500-nucleotide spacer sequence for preventing hairpin tangle formation. Conceivably, these miR-302 precursor sequences can be replaced by using miR-302-mimic siRNA and/or shRNA sequences for eliciting the same cell reprogramming functionality. Also, the uridine (U) content of these RNA sequences can be further replaced by pseudouridine and/or other modified nucleotides to increase the stability of the pre-miR-302 construct.

To overcome the low efficiency problem of hairpin-like RNA production, the pre-miR-302 and RdRp mRNA are preferably produced using a novel PCR-IVT methodology with an RNA polymerase-helicase mixture activity and an improved buffer system. Also, for increasing the efficiency of intracelluar delivery, the design pre-miR-302 and RdRp mRNA mixture composition can be further mixed, conjugated, encapsulated and/or formulated with at least a delivery agent selected from glycylglycerins, liposomes, nanoparticles, liposomal nanoparticles (LNP), conjugating molecules, infusion chemicals, gene gun materials, transposons, electroporation agents, and a combination thereof.

### BRIEF DESCRIPTION OF THE DRAWINGS

Referring particularly to the drawings for the purpose of illustration only and not limitation, there is illustrated:

FIG. 1 depicts the step-by-step procedure of the PCR-IVT methodology. For RNA production, a part or whole procedure of this PCR-IVT method can be adopted for either single or multiple cycle amplification of desired RNA products.

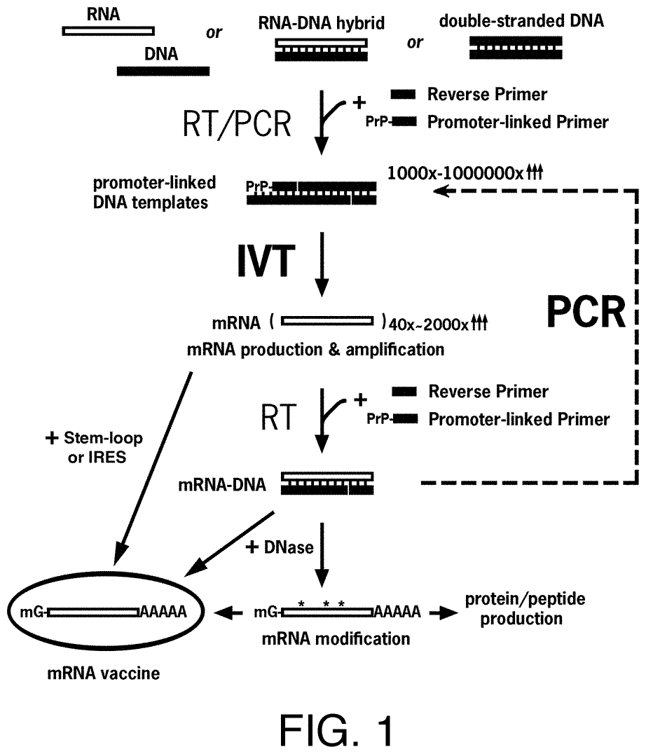

FIG. 2 depicts one of the preferred designs of the miR-302 precursor RNA (pre-miR-302) construct. At least an RNA-dependent RNA polymerase (RdRp)-binding site or more can be placed in either 5′-end or 3′-end region, or both, of the pre-miR-302 construct.

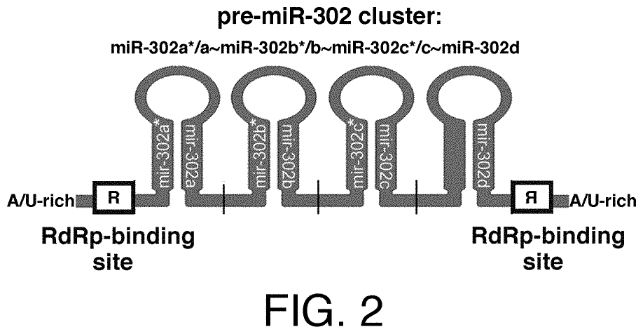

FIG. 3 shows markedly increased expressions of miR-302s (i.e. top to bottom: b, c, d, a) and RdRp mRNA in reprogrammed cells after co-transfection of both pre-miR-302 and RdRp mRNA (right) compared to that of transfection of only pre-miR-302 (middle), using Northern blot analysis.

FIG. 4 shows elevated expression of standard embryonic stem cell (ESC) markers such as Oct3/4, Sox2 and Nanog found in reprogrammed cells three days after co-transfection of pre-miR-302 and RdRp mRNA, using Western blot analysis.

FIG. 5 shows iPSC-derived embryoid body formation after co-transfection of pre-miR-302 and RdRp mRNA.

FIG. 6 shows global genomic DNA demethylation observed in the reprogrammed cells, similar to the status of human ESC genome.

FIG. 7 shows >92% high similarity of genome-wide gene expression patterns compared to those of human ESC WA01 (H1) and WA09 (H9) cell lines, using microarray analysis.

FIG. 8 shows iPSC-derived teratoma formation containing tissues derived from all three embryonic germ layers (ectoderm, mesoderm and definitive endoderm), using immunohisto-chemical staining with respective tissue cell markers.

### EXAMPLES

### 1. Human Cell Isolation and Cultivation

Starting tissue cells can be obtained from either enzymatically dissociated skin cells using Aasen's protocol (Nat. Protocols 5, 371-382, 2010) or simply from the buffy coat fraction of heparin-treated peripheral blood cells. The isolated tissue samples must be kept fresh and used immediately by mixing with 4 mg/mL collagenase I and 0.25% TrypLE for 15-45 min, depending on cell density, and rinsed by HBSS containing trypsin inhibitor two times and then transferred to a new sterilized microtube containing 0.3 mL of feeder-free SFM culture medium (IrvineScientific, CA). After that, cells were further dissociated by shaking in a microtube incubator for 1 min at 37° C. and then transferred the whole 0.3 mL cell suspension to a 35-mm Matrigel-coated culture dish containing 1 mL of feeder-free SFM culture medium supplemented with formulated pre-miR-302+RdRp mRNA mixture, LIF, and bFGF/FGF2, or other optional defined factors. The used concentrations of pre-miR-302+RdRp mRNA mixture, LIF, bFGF/FGF2, and other optional defined factors is ranged from 0.1 to 500 microgram (μg)/mL, respectively, in the cell culture medium. The cell culture medium and all of the supplements must be refreshed every 2˜3 days and the cells are passaged at about 50%˜60% confluence by exposing the cells to trypsin/EDTA for 1 min and then rinsing two times in HBSS containing trypsin inhibitor. For ASC expansion, the cells were replated at 1:5˜1:500 dilution in fresh feeder-free MSC Expansion SFM culture medium supplemented with formulated pre-miR-302+RdRp mRNA mixture, LIF, bFGF/FGF2, and/or other optional defined factors. For culturing keratinocytes, cells are isolated from skin tissues and cultivated in EpiLife serum-free cell culture medium supplemented with human keratinocyte growth supplements (HKGS, Invitrogen, Carlsbad, Calif.) in the presence of proper antibiotics at 37° C. under 5% CO₂. Culture cells are passaged at 50%-60% confluency by exposing cells to trypsin/EDTA solution for 1 min and rinsing once with phenol red-free DMEM medium (Invitrogen), and the detached cells are replated at 1:10 dilution in fresh EpiLife medium with HKGS supplements. Human cancer and normal cell lines A549, MCF7, PC3, HepG2, Colo-829 and BEAS-2B were obtained either from the American Type Culture Collection (ATCC, Rockville, Md.) or our collaborators and then maintained according to manufacturer's or provider's suggestions. After reprogramming, the resulting iPS cells (iPSCs) were cultivated and maintained following either Lin's feeder-free or Takahashi's feeder-based iPSC culture protocols (Lin et al., RNA 14:2115-2124, 2008; Lin et al., Nucleic Acids Res. 39:1054-1065, 2011; Takahashi K and Yamanaka S, Cell 126:663-676, 2006).

### 2. In-Vitro RNA Transfection

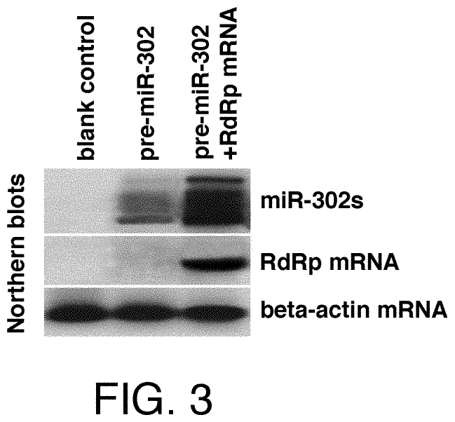

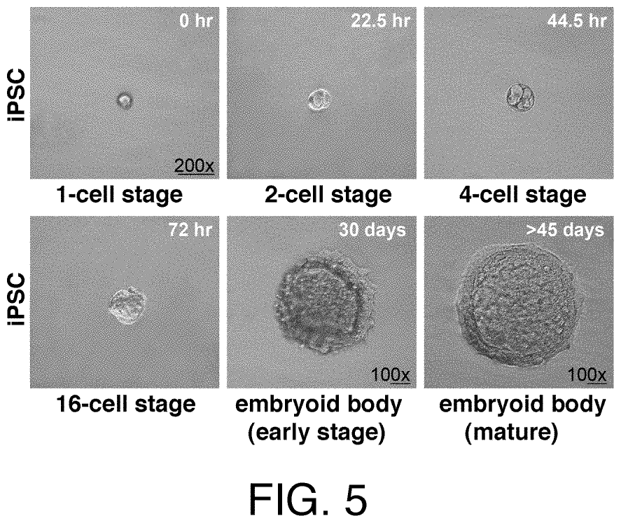

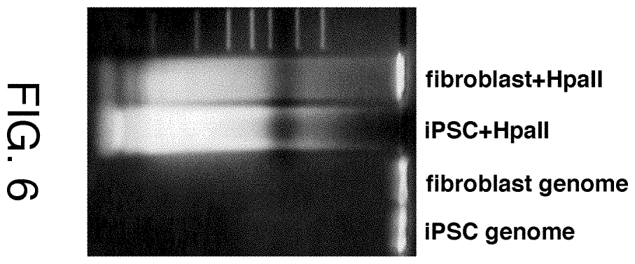

For RNA transfection, 0.5˜500 μg of isolated pre-miR-302 and RdRp mRNA mixture (ratio ranged from about 20:1 to 1:20) is dissolved in 0.5 ml of fresh cell culture medium and mixed with 1˜50 μl of In-VivoJetPEI or other similar transfection reagents. After 10˜30 min incubation, the mixture is then added into a cell culture containing 50%-60% confluency of the cultivated cells. The medium is reflashed every 12 to 48 hours, depending on cell types. This transfection procedure may be performed repeatedly to increase transfection efficiency. The transfection results are further analyzed by various detection methods and shown in FIG. 3 to FIG. 8, respectively.

### 3. DNA Demethylation Assay

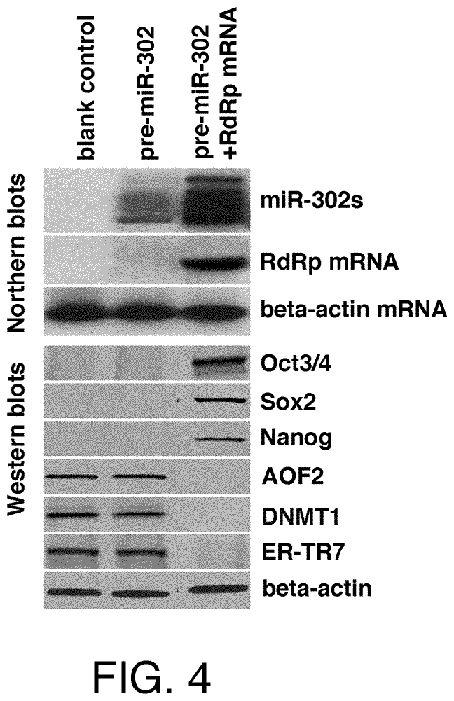

Genomic DNAs were isolated from about two million cells with a Roche DNA isolation kit (Sigma-Aldrich, MO, USA). About 2 microgram (μg) of the isolated genomic DNAs was digested with a CCGG-cutting restriction enzyme, HpaII, and then assessed with 1% agarose gel electrophoresis to determine genome-wide demethylation (FIG. 4).

### 4. Genome-Wide Microarray Analysis of Global Cellular Gene Expression Patterns

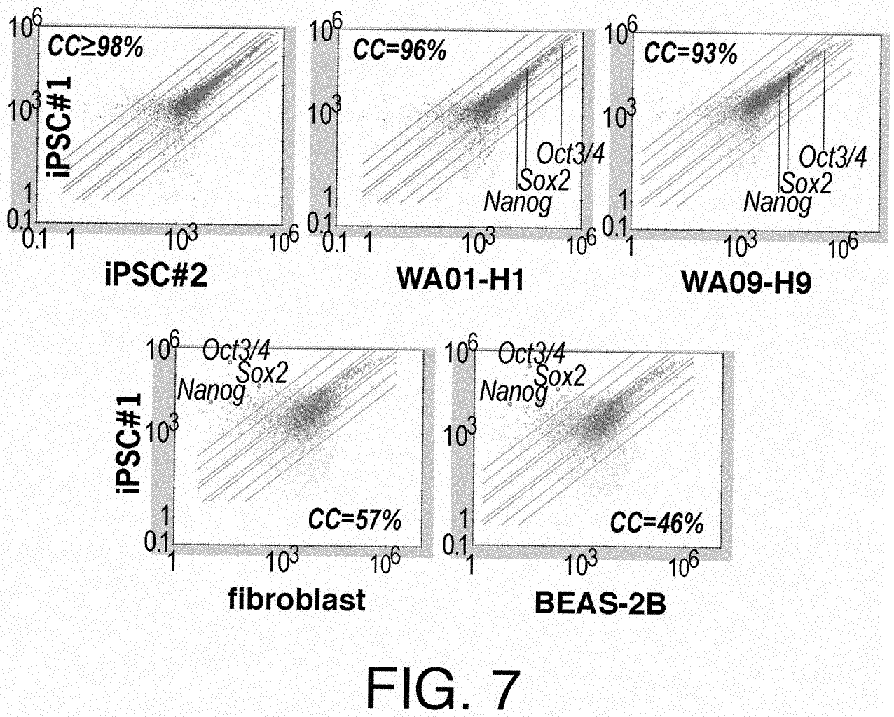

Human genome GeneChip arrays (Affymetrix, CA) were used to detect the expression patterns of genome-wide 47,000 human gene transcripts in iPS cells, as shown in FIG. 7. Each sample was tested in triplicate and the same experiment was repeated for four times. Total RNAs from each tested sample were isolated using RNeasy spin columns (Qiagen, MD). To prepare labeled probes for microarray hybridization, the extracted total RNAs (2 μg) were converted into cDNAs, using a T7 promoter-containing oligo(dT)₂₄ primer and SuperScript double-stranded cDNA synthesis kit (ThermoFisher Scientific, MA), following manufacturer's protocols. The resulting cDNAs were purified by phenol-chloroform extractions, precipitated with ethanol, and resuspended at a concentration of 0.5 μg/μl in diethyl pyrocarbonate (DEPC)-treated ddH₂O. Then, in-vitro transcription was performed, containing 1 μg of the dsDNAs, 7.5 mM unlabeled ATP and GTP, 5 mM unlabeled UTP and CTP, and 2 mM biotin-labeled CTP and UTP (biotin-11-CTP, biotin-16-UTP, Enzo Diagnostics), and 20 U of T7 RNA polymerase. Reactions were carried out for 4 hours at 37° C., and the resulting cRNAs were purified by RNeasy spin columns (Qiagen). A part of the cRNA sample was separated on a 1% agarose gel to check the size range, and then 10 μg of the cRNAs were fragmented randomly to an average size of 50 bases by heating at 94° C. for 35 min in 40 mM Tris-acetate, pH 8.0, 100 mM KOAc/30 mM MgOAc. Hybridizations were completed in 200 μl of AFFY buffer (Affymetrix, CA) at 40° C. for 16 hours with constant mixing. After hybridization, arrays were rinsed three times with 200 μl of 6×SSPE-T buffer (1×0.25 M sodium chloride/15 mM sodium phosphate, pH 7.6/1 mM EDTA/0.005% Triton) and then washed with 200 μl of 6×SSPE-T for 1 hour at 50° C. The arrays were further rinsed twice with 0.5×SSPE-T and washed with 0.5×SSPE-T at 50° C. for 15 min. Then, staining assays were done with 2 μg/ml streptavidin-phycoerythrin (Invitrogen) and 1 mg/ml acetylated BSA (Sigma-Aldrich) in 6×SSPE-T (pH 7.6). The arrays were read at 7.5 μm with a confocal scanner (Molecular Dynamics).

To identify the background variations, we duplicated the microarray tests using the same sample and selected two hundred genes, which were slightly presented in one side of the tests, for further comparison. The sample signals were normalized using the total average difference between perfectly matched probes and mismatched probes. Then, alterations of overall genome-wide gene expression patterns were analyzed using Affymetrix Microarray Suite version 5.0, Expression Console™ (Affymetrix) and Genesprings (Silicon Genetics) softwares. Changes in gene expression rates more than 1-fold were considered as positive differential genes. In gene clustering assays, a plug-in program Genetrix (Epicenter Software) was used in conjunction with the Affymetrix softwares. Signals of the sample were normalized with the internal house-keeping control average in each microarray. After normalization, as signal intensity increased from level 1 to level 65,535, the corresponding color changed from green to black, and to red. The level above 23,000 was considered to be a positive call in that a Northern blotting assay could positively detect.

5. Teratoma Formation and Guided iPSC Differentiation

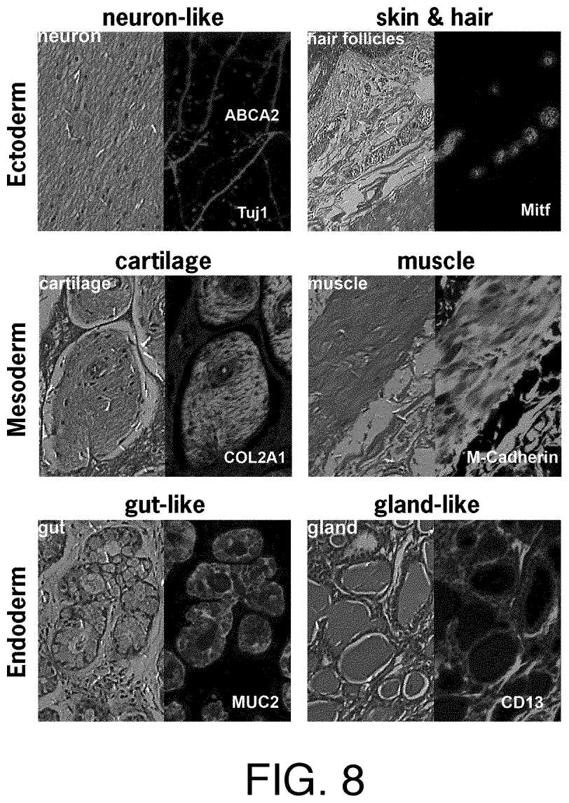

Xenograft transplantation of iPS-derived embryoid bodies into the uterus or peritoneal cavity of a 6-week-old female pseudopregnant immunocompromised SCID-beige mouse formed teratoma-like cysts, containing tissues derived from all three embryonic germ layers (ectoderm, mesoderm and definitive endoderm) (FIG. 8). The use of immunocompromised nude mice was to provide an in vivo environment mimicking transplantation therapy. The pseudopregnant mice were made by intraperitoneally injection of 1 IU human menopausal gonadotrophin (HMG) for two days and then human chorionic gonadotrophin (hCG) for one more day. For In vitro guidance of stem cell differentiation into the germ line lineage, iPS cells were maintained on polyornithine/laminin-coated dishes in DMEM/F12 (1:1; high glucose) medium supplemented with charcoal-stripped 10% FBS, 4 mM L-glutamine, 1 mM sodium pyruvate, 5 ng/ml activin and 50 ng/ml dihydrotestosterone (DHT) for 12 hours, at 37° C. under 5% CO₂. Then the cells were trypsinized, washed with 1×PBS, and collected in four aliquots of chilled Matrigel (100 μl each) and one aliquots of 100 μl 1×PBS. Immediately after that, we transplanted the cells into the hind limb muscle, peritoneum, uterus, subcutaneous neck skin (with Matrigel) and tail vein (with PBS) of 6-week-old immunocompromised SCID-beige nude mice. The mice were anesthetized with diethyl ether during experimental processing.

### 6. Immunostaining Assay

Cell/Tissue samples are fixed in 100% methanol for 30 min at 4° C. and then 4% paraformaldehyde (in 1×PBS, pH 7.4) for 10 min at 20° C. After that, the samples are incubated in 1×PBS containing 0.1%˜0.25% Triton X-100 for 10 min and then washed in 1×PBS three times for 5 min. For immunostaining, primary antibodies were purchased from Invitrogen (CA, USA) and Sigma-Aldrich (MO, USA), respectively. Dye-labeled goat anti-rabbit or horse anti-mouse antibody are used as the secondary antibody (Invitrogen, CA, USA). Results are examined and analyzed at 100× or 200× magnification under a fluorescent 80i microscopic quantitation system with a Metamorph imaging program (Nikon).

### 7. Novel PCR-IVT Protocol

Reverse transcription (RT) of desired mRNA is performed by adding about 0.01 ng-10 microgram (μg) of isolated mRNA into a 20˜50 μL RT reaction (SuperScript III cDNA RT kit, ThermoFisher Scientific, MA, USA), following the manufacturer's suggestions. Depending on the mRNA amount, the RT reaction mixture contains the mRNA, about 0.01˜20 nmole RT primer, a proper amount of dNTPs and reverse transcriptase in 1×RT buffer. Then, the RT reaction is incubated at 46˜65° C. for 1˜3 hours (hr), depending on the structure and length of the desired mRNA, so as to make at least a complementary DNA (cDNA) template for the next step of PCR. For cloning of viral RdRp cDNA, we design and use an RT-reverse primer 5′-GACAACAGGT GCGCTCAGGT CCT-3′ (SEQ.ID.NO.17) to perform RT of coronaviral COVID-19 mRNA into cDNA.

Next, polymerase chain reaction (PCR) is performed by adding about 0.01 pg˜10 μg of the RT-derived cDNAs into a 20˜50 μL PCR preparation mixture (High-Fidelity PCR kit, ThermoFisher Scientific, MA, USA), following the manufacturer's suggestions. Then, the PCR mixture is first incubated in five to twenty (5˜20) cycles of denaturation at 94° C. for 1 mim, annealing at 30˜55° C. for 30 sec-1 min, and then extension at 72° C. for 1-3 min, depending on the structure and length of the desired DNA and primers. After that, another ten to twenty (10˜20) cycles of PCR are performed with a series of sequential steps of denaturation at 94° C. for 1 mim, annealing at 50˜55° C. for 30 sec, and then extension at 72° C. for 1-3 min, depending on the structure and length of the resulting PCR products. Finally, the resulting PCR products are used as templates for IVT. For IVT template preparation, we design and use a specific pair of PCR primers for amplifying RNA promoter-containing RdRp cDNA templates from coronaviral mRNA, including SEQ.ID.NO.17 and 5′-GATATCTAAT ACGACTCACT ATAGGGAGAG GTATGGTACT TGGTAGTT-3′ (SEQ.ID.NO.18). Later, a 5′-cap nucleotide may be further incorporated in the resulting mRNA products during or after IVT. On the other hand, we also design and use another pair of PCR primers for amplifying RNA promoter-containing miR-302 familial cluster cDNA templates from isolated human cell genome DNAs, including 5′-GATATCTAAT ACGACTCACT ATAGGGAGAT CTGTGGGAAC TAGTTCAGGA AGGTAA-3′ (SEQ.ID.NO.19) and 5′-GTTCTCCTAA GCCTGTAGCC AAGAACTGCA CA-3′ (SEQ.ID.NO.20). In the designed primers, various RNA promoter sequences can be used, such as T7, T3 and/or SP6 promoter, and at least a RdRp initiation/binding site has been incorporated in the 5′-forward and 3′-reverse primer sequences, respectively. Also, the resulting miR-302 familial cluster template contains the cDNA sequences of at least one pre-miR-302a (SEQ.ID.NO.13), pre-miR-302b (SEQ.ID.NO.14), pre-miR-302c (SEQ.ID.NO.15), and/or pre-miR-302d (SEQ.ID.NO.16). Multiple same or different pre-miR-302 sequences can be repeatedly placed in the miR-302 familial cluster up to 9 hairpin-structured sequences.

For mRNA production, since a promoter-primer has been incorporated into the resulting PCR products, an improved IVT reaction can be performed to amplify desired mRNA sequences, using the PCR products as templates. The IVT reaction mixture contains 0.01 ng-10 μg of the PCR product, 0.1˜10 U of isolated coronaviral helicase (i.e. from COVID-19), a proper amount of NTPs and RNA polymerase (i.e. T7, T3, or SP6) in 1× transcription buffer. The contents of 1× transcription buffer may be adjusted according to the used RNA polymerase, following the manufacturer's suggestions. Additionally, the 1× transcription buffer may further conatin 0.001˜10 mM of betaine (trimethylglycine, TMG), dimethylsulfoxide (DMSO), and/or 3-(N-morpholino)propane sulfonic acid (MOPS), and/or a combination thereof, which facilitates the denaturation of highly structured RNA/DNA sequences, such as hairpins and stem-loop structures. Then, the IVT reaction is incubated at 37° C. for 1-6 hr, depending on the stability and activity of the used RNA polymerase(s). In this improved novel IVT reaction, at least an additional helicase enzyme is added in order to facilitate the unwinding of RNA/DNA secondary structures, such as hairpin-like stem-loop structures, so as to overcome the low efficiency problem of hairpin-like RNA production in vitro. Notably, the helicase enzyme can unwind the secondary structures in both DNA and RNA strands.

### 8. RNA Purification and Northern Blot Analysis

Desired mRNAs (10 μg) are isolated with a mirVana™ RNA isolation kit (Ambion, Austin, Tex.), following the manufacturer's protocol, and then further purified by using either 15% TBE-urea polyacrylamide gel or 3.5% low melting point agarose gel electrophoresis. For Northern blot analysis, the gel-fractionated mRNAs are electroblotted onto a nylon membrane. Detection of the mRNA and its IVT template (the PCR product) is performed with a labeled [LNA]-DNA probe complementary to a desired target sequence of the mRNA. The probe is further purified by high-performance liquid chromatography (HPLC) and tail-labeled with terminal transferase (20 units) for 20 min in the presence of either a dye-labeled nucleotide analog or [³²P]-dATP (>3000 Ci/mM, Amersham International, Arlington Heights, Ill.).

### 9. Protein Extraction and Western Blot Analysis

Cells (10⁶) are lysed with a CelLytic-M lysis/extraction reagent (Sigma) supplemented with protease inhibitors, Leupeptin, TLCK, TAME and PMSF, following the manufacturer's suggestion. Lysates are centrifuged at 12,000 rpm for 20 min at 4° C. and the supernatant is recovered. Protein concentrations are measured using an improved SOFTmax protein assay package on an E-max microplate reader (Molecular Devices, CA). Each 30 μg of cell lysate are added to SDS-PAGE sample buffer under reducing (+50 mM DTT) and non-reducing (no DTT) conditions, and boiled for 3 min before loading onto a 6-8% polyacylamide gel. Proteins are resolved by SDS-polyacrylamide gel electrophoresis (PAGE), electroblotted onto a nitrocellulose membrane and incubated in Odyssey blocking reagent (Li-Cor Biosciences, Lincoln, NB) for 2 hr at room temperature. Then, a primary antibody is applied to the reagent and incubated the mixture at 4° C. After overnight incubation, the membrane is rinsed three times with TBS-T and then exposed to goat anti-mouse IgG conjugated secondary antibody to Alexa Fluor 680 reactive dye (1:2,000; Invitrogen—Molecular Probes), for 1 hr at the room temperature. After three additional TBS-T rinses, fluorescent scanning of the immunoblot and image analysis are conducted using Li-Cor Odyssey Infrared Imager and Odyssey Software v.10 (Li-Cor).

### 10. In Vivo Transfection Assay

Isolated pre-miR-302 and RdRp mRNA mixture is mixed well with a proper amount of delivery agent, such as an In-VivoJetPEI transfection reagent, following the manufacturer's protocol, and then injected into blood veins or muscles of an animal, depending the purpose of applications. The delivery agent is used for mixing, conjugating, encapsulating or formulating the isolated pre-miR-302 and RdRp mRNA mixture, so as to not only protect the RNA contents from degradation but also facilitate the delivery of the isolated pre-miR-302 and RdRp mRNA mixture into specific target cells of interest in vitro, ex vivo and/or in vivo.

### 11. Statistic Analysis

All data were shown as averages and standard deviations (SD). Mean of each test group was calculated by AVERAGE of Microsoft Excel. SD was performed by STDEV. Statistical analysis of data was performed by One-Way ANOVA. Tukey and Dunnett's t post hoc test were used to identify the significance of data difference in each group. p<0.05 was considered significant (SPSS v12.0, Claritas Inc).

### REFERENCES

1. European Patent No. EP2198025B1 to Shi-Lung Lin et al.
2. U.S. Pat. No. 7,662,791 to Shi-Lung Lin et al.
3. U.S. Pat. No. 8,080,652 to Shi-Lung Lin et al.
4. U.S. Pat. No. 8,372,969 to Ying S Y and Shi-Lung Lin.
5. U.S. Pat. No. 8,609,831 to Shi-Lung Lin and Ying S Y.
6. U.S. Pat. No. 9,567,591 to Shi-Lung Lin et al.
7. U.S. Pat. No. 9,637,747 to Shi-Lung Lin et al.
8. U.S. Pat. No. 9,783,811 to Shi-Lung Lin et al.
9. U.S. Pat. No. 8,048,999 to Yamanaka et al.
10. U.S. Pat. No. 8,058,065 to Yamanaka et al.
11. U.S. patent application Ser. No. 15/661,346 to Shi-Lung Lin et al.
12. U.S. patent application Ser. No. 16/135,723 to Shi-Lung Lin et al.
13. U.S. patent application Ser. No. 17/489,357 to Shi-Lung Lin et al.
14. Shi-Lung Lin et al.; Mir-302 reprograms human skin cancer cells into a pluripotent ES-cell-like state. RNA 14:2115-2124, 2008.
15. Shi-Lung Lin et al.; MicroRNA miR-302 inhibits the tumorigenecity of human pluripotent stem cells by coordinate suppression of CDK2 and CDK4/6 cell cycle pathways. Cancer Res. 70:9473-9482, 2010.
16. Shi-Lung Lin et al.; Regulation of somatic cell reprogramming through inducible mir-302 expression. Nucleic Acids Res. 39:1054-1065, 2011.
17. Shi-Lung Lin and Ying S Y; Mechanism and method for generating tumor-free iPS cells using intronic microRNA miR302 induction. Shao-Yao Ying (Ed.) MicroRNA Protocols, 2nd Ed. Chapter 23, pp 295-324, Springer Publishers press, New York, 2012.
18. Shi-Lung Lin; Deciphering the mechanism behind induced pluripotent stem cell generation. Stem Cells 29:1645-1649, 2011.
19. Takahashi K and Yamanaka S; Induction of pluripotent stem cells from mouse embryonic and adult fibroblast cultures by defined factors. Cell 126:663-676, 2006.
20. Miyoshi et al.; Reprogramming of mouse and human cells to pluripotency using mature microRNAs. Cell Stem Cell 8:633-638, 2011.
21. Anokye-Danso et al.; Highly efficient miRNA-mediated reprogramming of mouse and human somatic cells to pluripotency. Cell Stein Cell 8:376-388, 2011
22. Shi-Lung Lin and Ji H; cDNA library construction using in-vitro transcriptional amplification. Methods Mol Biol. 221:93-101, 2003.
23. McDowell et al.; Determination of intrinsic transcription termination efficiency by RNA polymerase elongation rate. Science 266:822-825, 1994.
24. Aasen et al.; Isolation and cultivation of human keratinocytes from skin or plucked hair for the generation of induced pluripotent stem cells. Nat. Protocols 5:371-382, 2010.

1. A novel RNA composition for use in induced pluripotent stem cell (iPSC) generation, comprising:
A mixture of at least a miR-302 precursor RNA (pre-miR-302) construct and at least an RNA-dependent RNA polymerase (RdRp) mRNA, wherein the pre-miR-302 construct contains at least an RdRp binding site in its 5′-end or 3′-end region, or both, and wherein the RdRp mRNA is isolated or modified from RNA virus.

2. The composition as defined in claim 1, wherein the ratio of said pre-miR-302 and RdRp mRNA mixture is ranged from 20:1 to 1:20.

3. The composition as defined in claim 1, wherein said 5′-end RdRp binding site contains a sequence of either SEQ.ID.NO.1 or SEQ.ID.NO.2.

4. The composition as defined in claim 3, wherein said 5′-end RdRp binding site is selected from a sequence containing SEQ.ID.NO.3, SEQ.ID.NO.4, SEQ.ID.NO.5, or SEQ.ID.NO.6, or a combination thereof.

5. The composition as defined in claim 1, wherein said 3′-end RdRp binding site contains a sequence of either SEQ.ID.NO.7 or SEQ.ID.NO.8.

6. The composition as defined in claim 5, wherein said 3′-end RdRp binding site is selected from a sequence containing SEQ.ID.NO.9, SEQ.ID.NO.10, SEQ.ID.NO.11, or SEQ.ID.NO.12, or a combination thereof.

7. The composition as defined in claim 1, wherein said pre-miR-302 is selected from at least a sequence containing SEQ.ID.NO.13, SEQ.ID.NO.14, SEQ.ID.NO.15, or SEQ.ID.NO.16, or a combination thereof.

8. The composition as defined in claim 1, wherein said RdRp mRNA is isolated from RNA virus.

9. The composition as defined in claim 1, wherein said RdRp mRNA is coronaviral or hepatitis C viral RNA-dependent RNA polymerase mRNA.

10. The composition as defined in claim 1, wherein said pre-miR-302 is produced using a novel polymerase chain reaction-in-vitro transcription (PCR-IVT) methodology with an RNA polymerase and helicase mixture activity.

11. The composition as defined in claim 1, wherein said RdRp mRNA is produced using a novel polymerase chain reaction-in-vitro transcription (PCR-IVT) methodology with an RNA polymerase and helicase mixture activity.

12. The composition as defined in claim 10, wherein said helicase is an enzyme capable of unwinding both DNA and RNA secondary structures.

13. The composition as defined in claim 10, wherein the IVT reaction of said PCR-IVT methodology is performed in an improved buffer system containing 1× transcription buffer with additional 0.001˜10 mM of betaine (trimethylglycine, TMG), dimethylsulfoxide (DMSO), or 3-(N-morpholino)propane sulfonic acid (MOPS), or a combination thereof.

14. The composition as defined in claim 1, wherein said pre-miR-302 and RdRp mRNA mixture is further formulated with at least a delivery agent for facilitating intracellular transfection in vitro, ex vivo and/or in vivo.

15. The composition as defined in claim 14, wherein said delivery agent includes glycylglycerins, liposomes, nanoparticles, liposomal nanoparticles, conjugating molecules, infusion chemicals, gene gun materials, electroporation agents, transposon, and a combination thereof.

16. The composition as defined in claim 1, wherein said iPSCs can differentiate into various tissue cells derived from all three germ layers of ectoderm, mesoderm and endoderm.

17. The composition as defined in claim 16, wherein said iPSC-derived tissue cells are used for developing cell-based therapies.

18. The composition as defined in claim 1, wherein said iPSCs is used for developing stem cell-based therapies.

19. The composition as defined in claim 1, wherein said iPSCs is used for searching and/or producing new medicine materials.

20. The composition as defined in claim 1, wherein said pre-miR-302 and RdRp mRNA mixture is used for developing reprogramming-associated therapies and medicines.

21. The composition as defined in claim 1, wherein said pre-miR-302 and RdRp mRNA mixture is used as an ingredient in medicines or therapies.


SEQUENCE LISTING (20 sequences)

SEQ ID NO: 1 - RNA, 6 nt, Artificial Sequence
  Synthetic
ausugw

SEQ ID NO: 2 - RNA, 7 nt, Artificial Sequence
  Synthetic
ucwcywa

SEQ ID NO: 3 - RNA, 6 nt, Artificial Sequence
  Synthetic
aucugu

SEQ ID NO: 4 - RNA, 7 nt, Artificial Sequence
  Synthetic
ucucuaa

SEQ ID NO: 5 - RNA, 7 nt, Artificial Sequence
  Synthetic
ucuccua

SEQ ID NO: 6 - RNA, 5 nt, Artificial Sequence
  Synthetic
uucaa

SEQ ID NO: 7 - RNA, 6 nt, Artificial Sequence
  Synthetic
wcasau

SEQ ID NO: 8 - RNA, 6 nt, Artificial Sequence
  Synthetic
uwrgwr

SEQ ID NO: 9 - RNA, 6 nt, Artificial Sequence
  Synthetic
acagau

SEQ ID NO: 10 - RNA, 7 nt, Artificial Sequence
  Synthetic
uuagaga

SEQ ID NO: 11 - RNA, 7 nt, Artificial Sequence
  Synthetic
uaggaga

SEQ ID NO: 12 - RNA, 5 nt, Artificial Sequence
  Synthetic
uugaa

SEQ ID NO: 13 - RNA, 68 nt, Artificial Sequence
  Synthetic
ccuuugcuuuaacauggggguaccugcugugugaaacaaaaguaagugcuuccauguuucaguggagg

SEQ ID NO: 14 - RNA, 73 nt, Artificial Sequence
  Synthetic
gcucccuucaacuuuaacauggaagugcuuucugugacuuuaaaaguaagugcuuccauguuuuaguaggagu

SEQ ID NO: 15 - RNA, 69 nt, Artificial Sequence
  Synthetic
ccaccacuuaaacguggauguacuugcuuugaaacuaaagaaguaagugcuuccauguuuuggugaugg

SEQ ID NO: 16 - RNA, 68 nt, Artificial Sequence
  Synthetic
ccucuacuuuaacauggaggcacuugcugugacaugacaaaaauaagugcuuccauguuugagugugg

SEQ ID NO: 17 - DNA, 23 bp, Artificial Sequence
  Synthetic
gacaacaggtgcgctcaggtcct

SEQ ID NO: 18 - DNA, 48 bp, Artificial Sequence
  Synthetic
gatatctaatacgactcactatagggagaggtatggtacttggtagtt

SEQ ID NO: 19 - DNA, 56 bp, Artificial Sequence
  Synthetic
gatatctaatacgactcactatagggagatctgtgggaactagttcaggaaggtaa

SEQ ID NO: 20 - DNA, 32 bp, Artificial Sequence
  Synthetic
gttctcctaagcctgtagccaagaactgcaca